(19) United States
(12) Patent Application Publication
(10) Pub. No.: US 2008/0247985 A1
(43) Pub. Date: Oct. 9, 2008

(54) Composition base containing solubilized allantoin and urea for topical preparations

(76) Inventor: Jerry Zhang, Grayslake, IL (US)

Correspondence Address: JERRY ZHANG, 1061 CHADWICK DR., GRAYSLAKE, IL, 60030, US

(21) Appl. No.: 11/732,962

(22) Filed: Apr. 5, 2007

(51) Int. Cl. A61K 38/12; A61K 8/36; A61K 31/7048; A61K 31/455

(52) U.S. Cl. 424/78.03; 514/398; 514/476; 514/28; 514/11; 514/356

(57) ABSTRACT

Substantially homogeneous topical preparations for cosmetic, veterinary, and pharmaceutical use comprise at least one active ingredient solubilized in a novel composition base. The composition base comprises solubilized allantoin at a level of at least 0.5% and urea at a level of at least 10% in an aqueous vehicle.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to a novel composition base comprising solubilized allantoin and urea. Topical preparations may be formulated by incorporating at least one active ingredient into the composition base where the active ingredient is substantially solubilized.

### BACKGROUND OF THE INVENTION

Urea has been long recognized as a cosmetic ingredient in formulations acting as a humectant and moisturizer. High concentrations of urea, such as greater than 35%, are known to have keratolytic activity as well as mild, antimicrobial effect.

Allantoin, known for its therapeutic action on skin, has been widely used for decades in cosmetic and over-the-counter (OTC) topical formulations. It is also used in the topical pharmaceutical applications in skin ulcer therapy, psoriasis medications and analgesic gels. Important features of allantoin are keratolytic action and promotion and acceleration of cell proliferation. Allantoin is also used for its soothing and anti-irritating properties.

Allantoin has been classified by the Food and Drug Administration (FDA) OTC Topical Analgesic Review Panel as a Category I (safe and effective) active ingredient skin protectant at a level of 0.5% to 2%.

Although allantoin has been used extensively in cosmetic, dermatological, and pharmaceutical applications, solubility of allantoin at the FDA-approved levels is an issue. For example, solubility of allantoin in water at 25.degree. C. is 0.45%. In our co-pending U.S. patent application Ser. No. 11/542,326, which is incorporated herein by reference in its entirety, we disclosed that urea and urea derivatives are solubility enhancing agents being able to increase solubility of allantoin in water to at least 0.75%, at least 1.0%, and at least 2.0%.

For use as a cosmetic skin protectant or treatment of many dermal disorders, it is often preferable to use preparations containing solubilized active ingredients, such as a solution, spray or gel, rather than a cream, lotion or an ointment. Creams, lotions, (typically oil-in-water emulsions) and ointments (typically petroleum jelly based preparations) are often comedogenic, acnegenic, or less cosmetically appealing to patients. Furthermore, active ingredient is generally more bioavailable in solubilized form than in insoluble or suspended form. For medical conditions such as rosacea, or acne, it is often advantageous to use aqueous preparations with limited concentrations of oils, organic solvents, or surfactants. The aqueous preparations may minimize potential of further irritation caused by presence of oils, organic solvents or surfactants, to already inflamed and irritated skin. Many commercial products for treating rosacea and acne are aqueous preparations.

There is a need for enhancing efficacy of topical preparations for treating dermal disorders, such as, for example, rosacea and acne. There is also a need for a composition base into which other active ingredients can be incorporated to produce enhanced prophylactic or therapeutic efficacy.

### SUMMARY OF THE INVENTION

It has been unexpectedly discovered that substantially homogeneous topical preparations with enhanced prophylactic or therapeutic efficacy may be prepared by incorporating at least one active ingredient into a novel composition base. The novel composition base comprises solubilized urea at a level of at least 10% and allantoin at a level of at least 0.5% in an aqueous vehicle.

Accordingly, it is an object of the invention to formulate a preparation comprising at least one active ingredient solubilized in a novel composition base. The composition base comprises solubilized allantoin at a level of at least 0.5% and urea at a level of at least 10% in an aqueous vehicle.

Another object of the invention is to formulate a cosmetic, veterinary, or pharmaceutical preparation comprising a safe and effective amount of at least one active ingredient, allantoin at a level of at least 0.5%, and urea at a level of at least 10% in an aqueous vehicle in which the active ingredient, allantoin, and urea are substantially solubilized.

A further object of the invention is to formulate topical preparations for treating dermal disorders comprising a mixture of a safe and effective amount of at least one active ingredient, allantoin at a level of at least 0.5%, and urea at a level of at least 10% in an aqueous vehicle.

The above preparations are substantially homogeneous and have pH values in the range of from 3.5 to 7.0.

Still other objects and advantages of the invention will, in part, be obvious and will, in part, be apparent from the following detailed description of the preferred embodiments.

### DETAILED DESCRIPTION OF THE PREFERRED EMBODIMENTS

In the disclosed embodiments, a substantially homogeneous preparation comprises at least one active ingredient solubilized in a novel composition base. The composition base comprises allantoin and urea solubilized in an aqueous vehicle.

The term, ‘homogeneous’, as used herein, will be understood by persons of ordinary skill in the art. If there are uses of the term which are not clear to persons of ordinary skill in the art given the context in which is used, ‘homogeneous’ will mean uniform throughout, or ‘single-phase’, or ‘one-phase’. A substantially homogeneous preparation is a ‘single-phase’ or ‘one-phase’ preparation in which all ingredients are substantially solubilized.

The term ‘allantoin’, when used in accordance with the present invention, means allantoin that is either prepared from synthetic method or isolated from natural source (e.g., from comfrey extract), either in admixture or in pure or substantially pure form. All morphological forms of allantoin, crystalline, semi-crystalline, and amorphous, are contemplated and within the scope of the present invention, either in admixture or in pure or substantially pure form. Furthermore, the stereoisomers of allantoin are also contemplated and within the scope of the present invention. The definition of allantoin in accordance with the present invention embraces all possible stereoisomers and their mixtures. It particularly embraces the racemic forms and the isolated optical isomers having the specified activity.

The term ‘dissolved’, ‘dissolving’, ‘solubilized’ or ‘solubilizing’, when used in accordance with the present invention, means that an ingredient is substantially solubilized in the aqueous preparation, and that the ingredient will not exist to any appreciable degree in the particulate, crystalline or droplet form in the preparation.

As used herein, the term ‘about’ will be understood by persons of ordinary skill in the art and will vary to some extent on the context in which is used. If there are uses of the term which are not clear to persons of ordinary skill in the art given the context in which is used, ‘about’ will mean up to plus or minus 10% of the particular term.

The term ‘pharmaceutically acceptable’, as used herein, refers to those compounds, materials, preparations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term, ‘enhance efficacy’, ‘enhanced efficacy’, or ‘enhancing efficacy’, as used in accordance with the present invention, means any, some, or all of the following definitions:

a. producing cosmetic, veterinary, or pharmacological effect in a faster and/or more profound fashion;

b. modulating release profile of the active ingredient solubilized in the preparation of the present invention in a controlled fashion to achieve desired cosmetic, veterinary, or pharmacological effect. The release profile includes, for example, a zero-order release profile, or pulsating release profile, or fast onset followed by zero-order release profile, or any desired type of release profile suitable for inducing a desired cosmetic benefit or treating a medical condition;

c. reducing potential of side effects and/or adverse reactions.

All percentages referred to in this specification are percentages by weight of the total preparation unless otherwise indicated.

Allantoin is a polar, heterocyclic organic compound with chemical formula: C₄H₆N₄O₃ and molecular weight of 158.12 [chemical name: (2,5-dioxo-4-imidazolidinyl) urea; or 5-ureidohydantoin]. It is a product of purine metabolism. Racemic forms of allantoin are generally obtained from chemical synthesis. Optically pure forms of allantoin can be obtained by chiral separation procedures from the synthetic racemic forms or isolation from natural sources.

The dissolved allantoin may be present in the preparation of the present invention in an amount of at least 0.5%, at least 0.75%, at least 1%, or even as much as 5%. Preferably the dissolved allantoin may be present in an amount of from 0.5% to 3%.

The dissolved urea may be present in the preparation of the present invention in an amount of at least 10%, at least 15%, at least 20%, or even as much as 50%. Preferably the dissolved urea may be present in an amount of from 15% to 45%.

The active ingredient may be any suitable chemical compound which is substantially water soluble. The term, ‘substantially water soluble’, as used herein, means that the chemical compound is substantially solubilized in the aqueous preparation in accordance with the present invention at a safe and effective level for inducing desired cosmetic, veterinary, or pharmacological effect. In addition to the active ingredient to be solubilized, the aqueous preparation comprises solubilized allantoin, urea, and other customary auxiliaries and additives, or even other solubilized active ingredients.

The active ingredient may be a cosmetic, veterinary, or pharmacologically active agent. The active ingredient may be a prodrug. Cosmetic, veterinary, or pharmacologically active agents that have not been discovered yet are also contemplated and within the scope of the present invention.

The active ingredient may be present in the preparation in different forms, depending on which form yields desired cosmetic, veterinary, or pharmacological effect. Thus, the active ingredient may be in its free base or acid form, or in the form of salts, esters, or any other pharmaceutically acceptable derivatives, or as components of molecular complexes.

All morphological forms of the active ingredient, crystalline, semi-crystalline, and amorphous, are contemplated and within the scope of the present invention, either in admixture or in pure or substantially pure form. Furthermore, the stereoisomers of the active ingredient are also contemplated and within the scope of the present invention. The definition of the active ingredient in accordance with the present invention embraces all possible stereoisomers and their mixtures. It particularly embraces the racemic forms and the isolated optical isomers having the specified activity.

Examples of the cosmetic agents include, but not limited to:

Water soluble vitamins, for example, tocopherol (vitamin E) phosphate, vitamin B.sub.2 and derivatives thereof, vitamin B.sub.3 and derivatives thereof, vitamin C and derivatives thereof, lipoic acid, and combinations of the suitable vitamins thereof;

Water soluble antioxidants, for example, flavonoids, carnosine and derivatives thereof, glutathione, cysteine and derivatives thereof, proline, carnitine and derivatives thereof, and combinations of the suitable antioxidants thereof;

Skin whitening agent, for example, kojic acid, arbutin;

Combinations of the suitable cosmetic agents may be used in the preparations of the present invention provided that such combinations offer cosmetic benefits.

Examples of the pharmacologically active agents include, but not limited to:

Cardioactive agents, for example, organic nitrates such as nitroglycerin, isosorbide dinitrate, and isosorbide mononitrates; quinidine sulfate; thiazides such as chlorothiazide, and hydrochlorothiazide; adrenergic blockers, such as verapamil, dithiazem, and clonidine; and pharmaceutically acceptable salts thereof;

Retinoids, a “retinoid” is a keratolytic drug related to retinoic acid and generally includes chemical entities such as retinol and its esters and closely related naturally-occurring derivatives and structurally-related synthetic analogs. This includes, for example, retinol, retinal, tretinoin (all-trans retinoic acid), isotretinoin, adapalene (6-[3-(1-adamantyl)-4-methoxyphenyl]-2-naphthoic acid), and the like. Of these, tretinoin is preferred;

Analgesics and anesthetics, for example, lidocaine, tetracaine, dibucaine, prilocaine, benzocaine, and fentanyl, and pharmaceutically acceptable salts thereof;

Corticosteroid family of anti-inflammatory, anti-pruitic agents, for example, cortisone, desonide, and derivatives thereof;

Anti-viral agents, for example, acyclovir, and the like;

Anti-acne agents, for example, erythromycin, clindamycin, cephalosporins, and pharmaceutically acceptable salts or esters thereof, and niacinamide;

Anti-fungal agents, for example, amorolfine, clotrimazole, ketoconazole, miconazole, itraconazole, and fluconazole, and pharmaceutically acceptable salts thereof, terbinafine hydrochloride;

Skin cancer treatment agent, for example, fluorouracil;

Rosacea treatment agent, for example, metronidazole;

Hair growth agent, for example, minoxidil and pharmaceutically acceptable salts;

Hyperpigmentation treatment agent, for example, hydroquinone.

Additional examples of pharmacologically active agents include those compounds which are substantially water soluble and which are listed in the “Therapeutic Category and Biological Activity Index” of the Merck Index (12^{th} edition, 1996), the entire contents of which are thereby incorporated by reference.

The amount of the active ingredient in the preparation is a safe and effective amount for that compound. The term ‘safe and effective amount’, as used herein, means an amount of an active ingredient used in the preparations and methods of the present invention, sufficient enough to significantly and positively induce a cosmetic benefit or modify a condition to be treated, but low enough to avoid serious side effects, within the scope of sound medical advice.

The aqueous preparation, in accordance with the present invention, may contain conventional amounts of customary auxiliaries and additives provided that such auxiliaries and additives are physically and chemically compatible with the ingredients in the preparation, and do not substantially decrease the solubility of allantoin, urea, or active ingredient in the preparation, or reduce the cosmetic or therapeutic efficacy of the preparation. The term, ‘substantially decrease’, as used herein, means that inclusion of the auxiliaries and additives decreases the solubility of allantoin to less than 0.5%, or of urea to less than 10%, or of the active ingredient to a cosmetically or pharmacologically ineffective level, in the preparation.

Examples of customary auxiliaries and additives include, but not limited to: gelling agents, pH adjusting agents, moisturizing compounds, film-forming polymers, and other desirable ingredients. The customary auxiliaries and additives are substantially solubilized in the preparation of the present invention.

The aqueous preparation, in accordance with the present invention, may be in the form of a solution, spray or gel. Preferably the preparation is a gel. Therefore, the aqueous preparation preferably contains a gelling agent. Any gelling agent that is dispersible in the aqueous vehicle and forms an aqueous gel of substantially uniform consistency is suitable for use in the present invention.

Examples of the suitable gelling agents are polycarbohydrate based gelling agents and polyacrylic acid based gelling agents. Examples of the suitable polycarbohydrate gelling agents are hydroxyethylcellulose, hydroxypropylcellulose, and xanthan gum. Examples of the suitable polyacrylic acid gelling agents are CARBOPOL Brand 934, 940, 941, Ultrez 10, and Ultrez 20 (available from Noveon Corp., Cleveland, Ohio). Combinations of the polycarbohydrate gelling agents and/or polyacrylic acid gelling agent are also suitable as the gelling agents.

Allantoin and urea may undergo chemical degradation under basic conditions because of the presence of hydrolysable amide bonds in these molecules. Thus, pH of the preparation of the present invention is preferably kept at or below 7.0. Any pharmaceutically acceptable inorganic or organic acid or base may be used to adjust pH of the preparation. If the preparation is acidic, for example, pH is less than 3.5; the base may be used to adjust the pH.

If the preparation is basic, for example, pH is greater than 7.0; the acid may be used to adjust the pH. The pH of the aqueous preparation may be kept in the range of from about 3.5 to about 7.0, more preferably from about 4.0 to about 6.5, even more preferably from about 4.5 to about 6.0.

The aqueous preparation of the present invention may contain moisturizing compounds including, but not limited: polyhydric alcohols (also known as polyols), polyol ethers and esters, low molecular weight polyethylene glycols, lactates, sugars, methyl glucose ethers, sodium pyrrolidone carboxylic acid, sodium hyaluronate, N-hydroxyethyl urea, panthenol, hyaluronic acid, .alpha.- and .beta.-hydroxy acids, or combinations of the suitable moisturizing compounds.

Examples of the suitable polyols are glycerin (also known as glycerol), propylene glycol (also known as 1,2-propanediol), 1,3-propanediol, 1,2-butanediol, 1,3-butanediol, 1,4-butanediol, 2,3-butanediol, 1,2-pentanediol, 1,5-pentanediol, 1,2-hexanediol, 2-methyl-2,4,-pentanediol (also known as hexylene glycol), 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Preferred polyols are glycerin, propylene glycol, 1,4-butanediol, and hexylene glycol. Examples of the suitable low molecular weight polyethylene glycols (PEG) are PEG-4, PEG-6, PEG-8, and PEG-10. Examples of the suitable lactates are ammonium lactate, sodium lactate, and potassium lactate. Examples of the suitable methyl glucose ethers are methyl gluceth-10 and methyl gluceth-20. Examples of the suitable .alpha.- and .beta.-hydroxy acids are glycolic acid, lactic acid, mandelic acid, and salicylic acid.

The film forming properties of polymers help maintain active ingredients on the site of application, which may help enhance prophylactic or therapeutic efficacy. Examples of the suitable film forming polymers are hydroxypropyl cellulose, polyvinylpyrrolidone, polyvinylpyrrolidone vinylacetate copolymer, polyvinyl alcohol, and combinations thereof.

The film forming polymers may be added to the preparation of the present invention in an amount of from about 0.1% to about 40%, preferably from 0.5% to about 20%.

The aqueous preparation of the present invention may also contain one or more other desirable ingredients including, but not limited to: skin penetration enhancers, herbal extracts, chelating agents, preservatives, colorants, fragrances, and so on. Examples of the suitable herbal extracts are grape seed extract and onion extract. Examples of the suitable skin penetration enhancers are ethanol, isopropanol, dimethyl isosorbide, and N-methyl-1-pyrrolidone. Examples of the suitable chelating agents are EDTA (ethylenediaminetetraacetic acid), EGTA [ethylenebis(oxyethylenenitrilo)tetraacetic acid], and pharmaceutically acceptable salts thereof.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

This example is to demonstrate that the topical preparation formulated in the novel composition base of the present invention shows enhanced efficacy. The active ingredient is niacin, a rubefacient.
Component Amount (weight percentage)
Formulation 1A (present invention):
Allantoin 0.75%
Urea 15%
Propylene glycol 5%
Niacin 0.5%
Hydroxyethyl cellulose 1.0%
Water 77.75%
Formulation 1B (without solubilized allantoin, for comparison):
Urea 15%
Propylene glycol 5%
Niacin 0.5%
Hydroxyethyl cellulose 1.0%
Water 78.5%
Formulation 1C (without solubilized urea and allantoin, for comparison):
Propylene glycol 5%
Niacin 0.5%
Hydroxyethyl cellulose 1.0%
Water 93.5%

Formulation 1A was prepared by adding urea and allantoin into a mixture of propylene glycol and water. Then, the mixture was heated to a temperature of about 40.degree. C. to facilitate solubilization of urea and allantoin while stirring until dissolved. The solution was allowed to cool to room temperature. Niacin was added and dissolved. Hydroxyethyl cellulose (tradename: Natrosol CS 250HR from Hercules Inc.) was shifted into the solution. The mixture was kept stirring until the hydroxyethyl cellulose was gelled. A clear gel was formed.

Formulations 1B and 1C were prepared in a similar procedure. Clear gels were formed in both cases.

Niacin is a known rubefacient. When applied to skin, flushing (reddening in skin color) might be observed, depending on amount of niacin used. Thus, niacin assay was used as a visualization tool to evaluate and compare efficacies of the topical preparations.

Formulations 1A, 1B and 1C were applied to three areas of volar forearm of a human subject. The application sites were circles of about 2 centimeters in diameter and about 4 centimeters border-to-border from each other. About 50 mg of each formulation was applied. The product was evenly spread over the entire site using a glass rod. Onset time of flushing (reddening in skin color) was recorded. The onset of skin flushing is defined as reddening in skin color with clear outline of the application site and intense in color. The tests were done in duplicates on both arms of the human subject. The average onset time in minutes are listed below.
Formulation 1A 1B 1C
Average onset time (min) 25 33 65

This result suggests a faster onset time for the formulation prepared in the composition base in accordance with the present invention. The active ingredient solubilized in the composition base exhibits enhanced efficacy (faster rubefacient effect).

### EXAMPLE 2

This example is to demonstrate physical stability of the preparation in accordance with the present invention. Niacinamide, a water soluble vitamin in vitamin B.sub.3 family, is used as the active ingredient.

| Component | Amount (weight percentage) |
|---|---|
| Allantoin | 1% |
| Urea | 20% |
| Niacinamide | 5% |
| Tetrasodium EDTA | 0.15% |
| Citric acid | q.s pH about 5.5 |
| Germaben - II | 0.5% |
| Propylene glycol | 5% |
| PEG-6 | 3% |
| Hydroxyethyl cellulose | 1.25% |
| Water | q.s 100% |

Urea and allantoin were added to water at room temperature. The mixture was heated to a temperature of about 40.degree.C. while stirring until urea and allantoin were completely dissolved. The solution was cooled to room temperature. Niacinamide and tetrasodium EDTA were added to the solution and dissolved. The pH of the solution was adjusted to about 5.5 using citric acid. Propylene glycol, Germaben-II, and PEG-6 were added and solubilized. Germaben-II is an antimicrobial preservative system consisting of propylene glycol and diazolidinyl urea and methylparaben and propylparaben (available from ISP Corp. Wayne, N.J.). Add water to q.s. the batch to final weight. Hydroxyethyl cellulose (Natrosol CS 250HR) was shifted into the solution. The mixture was kept stirring until gelled. A clear gel was formed.

The gel was packaged in a clear glass vial. The glass vial was sealed and placed in a refrigerator maintained at a temperature of about 4.degree. C. for 7 days. After 7 days, the vial was removed from the refrigerator. It was found that the gel remained clear and no crystallization or precipitation was evident in the gel.

### EXAMPLE 3

This example is to formulate a rosacea treatment gel in accordance with the present invention. Metronidazole is used as the active ingredient.

| Component | Amount (weight percentage) |
|---|---|
| Allantoin | 1% |
| Urea | 20% |
| Metronidazole | 1% |
| Germaben - II | 0.5% |
| Propylene glycol | 5% |
| PEG-6 | 3% |
| Hydroxyethyl cellulose | 1.25% |
| Water | 68.25% |

Urea, allantoin, and metronidazole were added to water at room temperature. The mixture was heated to a temperature of about 40.degree.C. while stirring until urea, allantoin, and metronidazole were completely dissolved. The solution was cooled to room temperature. The pH of the solution was about 5.8. Propylene glycol, Germaben-II, and PEG-6 were added and solubilized. Add water to q.s. the batch to final weight. Hydroxyethyl cellulose (Natrosol CS 250HR) was shifted into the solution. The mixture was kept stirring until gelled. A clear gel was formed. The gel is suitable for use to treat rosacea.

It will thus be seen that the objects set forth above, among those made apparent from the preceding description, are efficiently attained and, since certain changes may be made in carrying out the above process and in the preparation set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A substantially homogeneous preparation for topical application, comprising, by weight of the total preparation:
allantoin in an amount of from about 0.5% to about 5%,
urea in an amount of from about 10% to about 50%,
at least one active ingredient,
an aqueous vehicle,
wherein allantoin, urea, and the active ingredient are substantially solubilized in the preparation.

2. The preparation of claim 1 wherein allantoin is in an amount of from about 0.75% to about 3% and urea is in an amount of from about 15% to about 45%.

3. The preparation of claim 1 wherein allantoin is in an amount of from about 1% to about 2% and urea is in an amount of from about 20% to about 40%.

4. The preparation of claim 1 wherein the active ingredient is a cosmetic agent selected from the group consisting of water soluble vitamin, antioxidant, skin whitening agent, and combinations thereof.

5. The preparation of claim 1 wherein the active ingredient is a pharmacologically active agent selected from the group consisting of anti-acne agent, rosacea treatment agent, and anti-fungal agent.

6. The preparation of claim 5 wherein the anti-acne agent is selected from the group consisting of erythromycin, clindamycin, cephalosporins, and derivatives thereof, and niacinamide.

7. The preparation of claim 5 wherein the rosacea treatment agent is metronidazole.

8. The preparation of claim 1 wherein pH of the preparation is in the range of from about 3.5 to about 7.0.

9. The preparation of claim 1 wherein pH of the preparation is in the range of from about 4.5 to about 6.0.

10. The preparation of claim 1 wherein the active ingredient is metronidazole.

11. A topical preparation, comprising:
at least one active ingredient solubilized in a composition base, wherein said base comprising, by weight of the total preparation, (a) urea in an amount of from about 10% to about 50%, (b) allantoin in an amount of from about 0.5% to about 5%, (c) an aqueous vehicle, wherein said urea and allantoin are substantially solubilized in said vehicle,
wherein said preparation is substantially homogeneous.

12. The preparation of claim 11 wherein allantoin is in an amount of from about 0.75% to about 3% and urea is in an amount of from about 15% to about 45%.

13. The method of claim 11 wherein allantoin is in an amount of from about 1% to about 2% and urea is in an amount of from about 20% to about 40%.

14. The preparation of claim 11 wherein the active ingredient is a cosmetic agent selected from the group consisting of water soluble vitamin, antioxidant, skin whitening agent, and combinations thereof.

15. The preparation of claim 11 wherein the active ingredient is a pharmacologically active agent selected from the group consisting of anti-acne agent, rosacea treatment agent, and anti-fungal agent.

16. The preparation of claim 15 wherein the anti-acne agent is selected from the group consisting of erythromycin, clindamycin, cephalosporins, and derivatives thereof, and niacinamide.

17. The preparation of claim 15 wherein the rosacea treatment agent is metronidazole.

18. The preparation of claim 11 wherein pH of the preparation is in the range of from about 3.5 to about 7.0.

19. The preparation of claim 11 wherein pH of the preparation is in the range of from about 4.5 to about 6.0.

20. The preparation of claim 11 wherein the active ingredient is metronidazole.
